(19) United States
(12) Patent Application Publication
(10) Pub. No.: US 2024/0145064 A1
(43) Pub. Date: May 2, 2024

(54) Method For Conducting A Health, Wellness and Weight Loss Reality Show

(71) Applicant: Keith Marz, Sherman Oaks, CA (US)

(72) Inventor: Keith Marz, Sherman Oaks, CA (US)

(21) Appl. No.: 17/979,286

(22) Filed: Nov. 2, 2022

(51) Int. Cl. G16H 20/60

(52) U.S. Cl. CPC G16H 20/60

(57) ABSTRACT

A method for conducting a health, wellness and weight loss reality show that partners at least one participant with at least one host. The method has the participant going to a collaborating store and selecting various products for purchase and use. The host(s) accompanies the participant(s) and comments on/judges the selected products. The host(s) will point out the strengths and the weaknesses of the products from a health, wellness and weight loss potential and perspective. An alternate method has the host interact with the participant immediately after the participant leaves the store after shopping, or the host can visit the participant's dwelling and comment on judge existing products in the cupboards or refrigerator. Whichever method is employed, the goal is to allow an audience to watch as an average/common person selects and purchases, or possesses consumable products that could be detrimental to their health, wellness and optimal weight.

### TECHNICAL FIELD

The invention generally pertains to reality television shows, and more particularly to a reality show focused on health, wellness and weight loss strengths and weaknesses and applicable to products that a participant purchases or possesses.

### BACKGROUND ART

In the United States, as well as many other countries, one of the most popular forms of broadcast entertainment is a reality television show. Typically, these reality shows feature common people (i.e. non-actors) doing or performing various acts such as singing, losing weight or even just living in a shared space with other people.

The entertaining factor results in how the people on the show deal with or interact with various scenarios or individuals. One of the most important aspects is watching a seemingly ordinary/average person excel at something. Another factor is how an ordinary/average person can go from a negative situation to a positive situation, and in the process improving their lives.

For many reality shows there does appear to be a template for producing the show. For example, a group of individuals compete against each other to discover who is the best at a particular endeavor. Usually, there will be celebrity hosts or judges, as well as having the audience vote for their favorite contestant.

While this format is effective and fairly entertaining, there does appear to be a need for something new. An obvious suggestion would be to have participants and hosts would interact in some manner, and that the audience member could relate to and apply to their life. This could be accomplished by taking something that most people do and allowing a participant to follow their individual pattern, which would coincide with the same pattern as experienced by the audience.

A search of the prior art did not disclose any literature or patents that read directly on the claims of the instant invention. However, the following U.S. patents are considered related:

| PAT. NO. | INVENTOR | ISSUED |
|---|---|---|
| 5,860,862 | Junkin | Jan. 19, 1999 |
| 6,439,997 | Brasseur, et al | Aug. 27, 2002 |
| 9,064,370 | Daniel | Jun. 23, 2015 |
| 2012/0274773 | Daniel, et al | Nov. 1, 2012 |

The U.S. Pat. No. 5,860,862 discloses an interactive apparatus and method that allows participants to compete in an interactive game based on a real time event. A visual display or audio broadcast shown or broadcast on a device of an interactive apparatus with score values indicating the players' performance in the event. Using the interactive apparatus, the participants select and trade members of a team, information regarding the performance of the participant's team allows the participant to select and trade members that are listed in a roaster database while the event is occurring.

The U.S. Pat. No. 6,439,997 discloses a method for creating and providing information used in a television game show. The information is obtained from the Internet and usersregister and answer a questionnaire to create a profile. A fixed number of user profiles are randomly selected and posted on the Internet. Users then vote for their favorite user profile, and the winner is given an award, presented live during a television broadcast. Sponsors provide the award and will be given prominent advertisement.

The U.S. Pat. No. 9,064,370 discloses a method for conducting a reality sports television show. The method includes providing a cast that comprises a plurality of sports players that are undrafted, broadcasting private lives of the cast and broadcasting the cast playing at least one sports game that comprises a game without timeouts and huddles. An on-field communication between the cast is accomplished by using a means for in-game communications, which includes at least one player transceiver and at least one coach transceiver for audio or visual communications to and from players, coaches, referees, or television production members.

The 2012/0274773 publication discloses a method for conducting a reality golf television show, comprising conducting a selection process that comprises a first component based on participants' driving range ability, oral skills, attitude and golf knowledge, and a second component based on participants' golf course performance. The selection is performed by collecting votes from viewers conducting a golf tournament, and following each participant with a camera and recording their everyday lives.

For background purposes and indicative of the art to which the invention relates, reference may be made to the following remaining patents found in the patent search.

| PAT. NO. | INVENTOR | ISSUED |
|---|---|---|
| 5,600,730 | Kenning, et al | Feb. 4, 1997 |
| 5,890,963 | Yen | Apr. 6, 1999 |
| 8,337,312 | Jones | Dec. 25, 2012 |
| 2007/0207846 | Burack, et al | Sep. 6, 2007 |
| 2008/0161113 | Hansen, et al | Jul. 3, 2008 |
| 2012/0129610 | Mazursky, et al | May 24, 2012 |

### DISCLOSURE OF THE INVENTION

A method for conducting a health, wellness and weight loss reality show that partners at least one participant with at least one host. The method can be produced by multiple means. The first mems has the participant going to a collaborating store, such as a supermarket, and selecting various products for purchase and use. The host(s) accompanies the participant(s) and comments on/judges the selected products. The host(s) will point out the strengths, and (more frequently) the weaknesses of the individual products from a health, wellness and weight loss potential and perspective. For example, it a participant selects a breakfast cereal with a large amount of sugar, the host will make the participant aware of the sugar content and offer alternatives. Or if the participant does not select a minimal amount of fruits and vegetables the host will offer suggestions that could be used to improve the participants dietary/health needs.

An alternate means would have the host interact with the participant immediately after the participant leaves the store after shopping (such as a parking lot), or the host can visit the participant's dwelling and comment on/judge existing products in the cupboards or refrigerator. Which ever means is employed the goal of the method is the same to allow an audience to watch as an average/common person selects and purchases, or possesses consumable products that could be detrimental to their health, wellness and optimal weight. By having the host critique the participant on their consumable purchase, both the participants and the audience members can educate and informed about better u choices that can be made regarding food and other products. It is important to note that the host does not berate or insult the participant(s). Rather the critiques are made in a low-key, often humorous styles that is entertaining and educational for both the participants and the audience. Also, in addition to a host(s), the show could have a panel with at least two individuals who are knowledgeable in health fields and would comment on and judge the participant(s) actions along with the host(s), or would comment on and judge the participant(s) actions and the host(s) comments and suggestions.

In view of the above disclosure the primary object of the invention is to produce a method for conducting a healthy, wellness and weight loss reality show that aims to educate participants and audience members about the importance of knowing how healthy consumable products are and how choices can be altered to improve the overall healthiness of what common people typically purchase.

In addition to the primary object of the invention, it is also an object of the invention to produce a method for conducting a healthy, wellness and weight loss reality show that:
can be applied to adults and children,
is effective for individuals, couples and families,
does not require a significant investment or change in lifestyle,
allow for stores to obtain free advertising,
allows various products to be featured and the benefits of using certain products to be shown,
can product revenue from multiple sources,
does not require a large operating budget,
is easy to direct and produce,

These and other objects and advantages of the present invention will become apparent from the subsequent detailed description of the preferred embodiment and the appended claims taken in conjunction with the accompanying drawings.

### BRIEF DESCRIPTION OF THE DRAWINGS

FIG. 1 is a block diagram showing the steps of a first variation of a method for conducting a health, wellness and weight loss reality show,

FIG. 2 is a block diagram showing the steps of a second variation of a method for conducting a health, wellness and weight loss reality show.

FIG. 3 is a block diagram showing the steps of an alternate version of the second variation of the method for conducting a health, wellness and weight loss reality show.

### BEST MODE FOR CARRYING OUT THE INVENTION

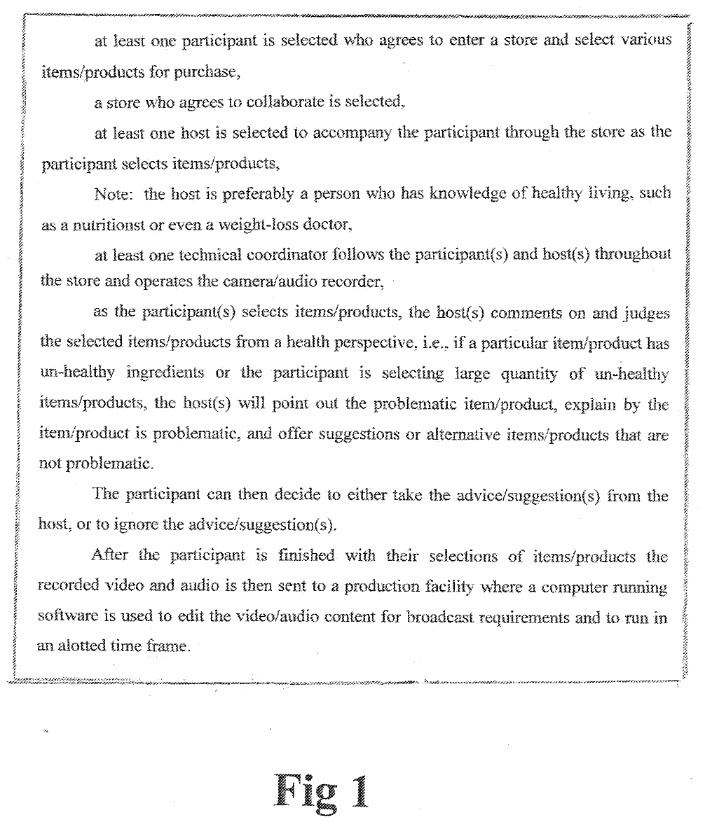

The best mode for carrying out the invention is presented in terms that disclose a preferred embodiment, with multiple variations of a method for conducting a health, wellness and weight loss reality show. One of the most popular types of broadcast entertainment are reality television shows. Typically, these shows feature a person or persons who is competing in a specific skill or goal, such as singing or losing weight, or will use “hidden” cameras showing a group of people in a certain situation, such as various people living together in a share environment. While these shows am, often entertaining, a reality show could also be utilized to provide education and life improvements skills in addition to the entertainment. The method for conducting a healthy, wellness and weight loss reality show 10 (show 10), as shown in FIGS. 1-3, accomplishes this exact goal.

The show 10 is disclosed herein with three variations, although other variations and modifications could be easily implemented. The first variation of the show is comprised of at least one participant who selects products for purchase from a collaborating store, at least one host who accompanies the at least one participant while the participant selects the products in the store, at least one participant transceiver for receiving and transmitting audio and visual communications to a production facility, at least one host transceiver for receiving and transmitting audio and visual communication to a production facility, and a computer operating software that produces the communication from the at least one participant and at least one host for broadcast on a viewing medium, with the host commenting on and judging the selections of the participant from a health, wellness and weight loss potential and perspective.

The term collaborating store can include a market or a supermarket, a big box retailer (such as Walmart, Costco or Sam's Club), a drug store or any location, retail or wholesale, where a person can purchase food items, beverages, or other personal or hygienic products. The word “collaborating” discloses that the store is aware of and gives permission for the show 10 to take place in and record/broadcast the show 10 from the store's premises. In return for collaboration the store will receive and benefit from significant exposure and advertising since the store itself will be a featured component of the show 10.

The transceivers will comprise at least one camera with audio capturing capability. The method the show 10 is conducted, as shown in FIG. 1, by the following steps:
at least one participant is selected who agrees to enter a store and select various items/products for purchase,
a store who agrees to collaborate is selected,
at least one host is selected to accompany the participant through the store as the participant selects items/products.

Note: the host is preferably a person who has knowledge of healthy living, such as a nutritionist or even a weight-loss doctor,
at least one technical coordinator follows the participant(s) and host(s) throughout the store and operates the camera/audio recorder,
as the participant(s) selects items/products, the host(s) comments on and judges the selected items/products from a health perspective, i.e., if a particular item/product has un-healthy ingredients or the participant is selecting large quantity of un-healthy items/products, the host(s) will point out the problematic item/product, explain by the item/product is problematic, and offer suggestions or alternative items/products that are not problematic.

The participant can then decide to either take the advice/suggestion(s) from the host, or to ignore the advice/suggestion(s).

After the participant is finished with their selections of items/products the recorded video and audio is then sent to a production facility where a computer running software is used to edit the video/audio content for broadcast requirements and to run in an alotted time frame.

A second variation of the show 10 is similar to the first variation and is comprised of:
at least one participant who has purchased products from a store,
at least one host who comments on and judges the purchased products,
at least one transceiver for receiving and transmitting audio and visual communication to a production facility, and
a computer opening software that process the communications from the at least one participant and the at least one host for broadcast on a viewing medium.

The major difference between the first and second variation is participant(s) and host(s) do not internet within a store, while the participant(s) selects items/products. Rather, the host(s) go to the participant(s) dwelling and views items/product that have been previously purchased and are present within the dwelling, such as food items in cupboards, pantries and refrigerators. The host(s) will see the items/product the participant(s) have purchased/acquired and will comment on and judge from a health, wellness and weight loss potential and perspective. The host(s) will also offer suggestions/advice on which alternative items/products can be purchased/acquired in the future, in lieu of the existing items/products.

Alternately, instead of going to a participant's dwelling, the host(s) (and technical coordinator with the camera/audio recorder) can interact with the participant(s) immediately after the participant(s) leave a store with the items/products purchased.

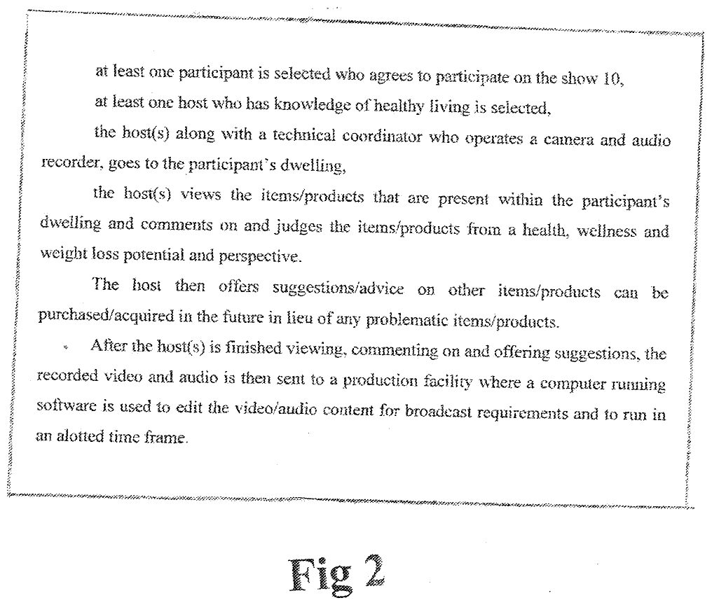

The method the show is conducted for the second variation, as shown in FIG. 2, by the following steps:
at least one participant is selected who agrees to participate on the show 10,
at least one host who has knowledge of healthy living is selected,
the host(s) along with a technical coordinator who operates a camera and audio recorder, goes to the participant's dwelling,
the host(s) views the items/products that are present within the participants dwelling and comments on and judges the items/products from a health, wellness and weight loss potential and perspective.
the host then offers suggestions/advice on other items/products that can be purchased/acquired in the future in lieu of any problematic items/products.
after the host(s) is finished viewing, commenting on and offering suggestions, the recorded video and audio is then sent to a production facility where a computer running software is used to edit the video/audio content for broadcast requirements and to run in an alotted time frame.

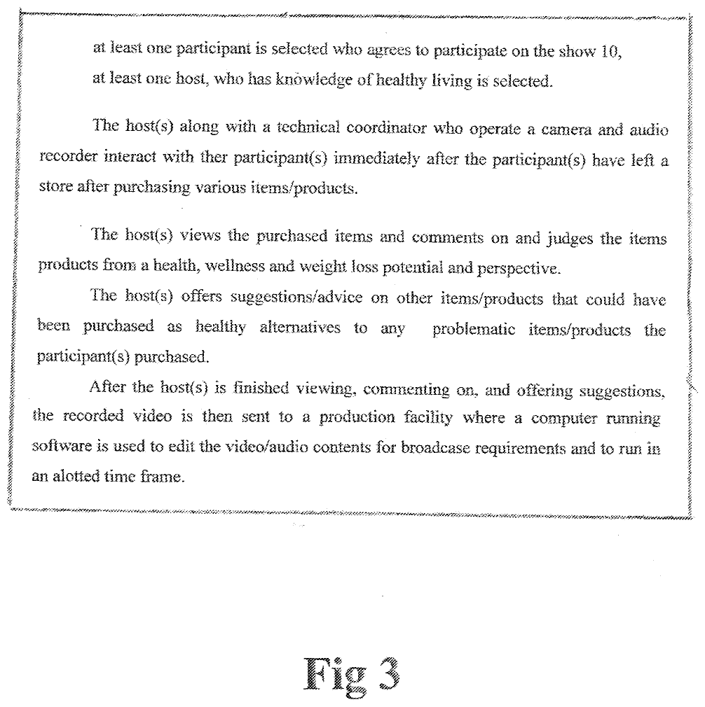

The alternative to the method of the second variation of the show 10, as shown in FIG. 3, is by the following steps:
at least one participant is selected who agrees to participate on the show 10,
at least one host, who has knowledge of healthy living is selected.
the host(s) along with a technical coordinator who operate a camera and audio recorder interact with the participant(s) immediately after the participant(s) have left a store after purchasing various items/products.
the host(s) views the purchased items and comments on and judges the items products from a health, wellness and weight loss potential and perspective.
the host(s) offers suggestions/advice on other items/products that could have been purchased as healthy alternatives to any problematic items/products the participant(s) purchased,
after the host(s) is finished viewing, commenting on, and offering suggestions, the recorded video is then sent to a production facility where a computer running software is used to edit the video/audio contents for broadcase requirements and to run in an alotted time frame.

Either of the two variations of the show 10 or the alternative can also have a panel comprised of at least two individuals who each have knowledge or experience in health related fields. The individuals comment on and judge the choices the participants make when shopping and/or the comments and judging of the host(s). When the panel is an element of the show, a viewing audience, either remote (i.e., at home) or in studio, can interact with the panel of individuals. Typically, the interaction will consist of commenting on and judging the comments and judgements the panel individuals put forth. Also, at least one member of the viewing audience can win a reward (or prize) based on the metrics of the show and the contribution(s) the audience member makes. For example, the audience member who suggests the best alternative to a certain product, as voted on by other audience members, the panel individuals or even the host(s), would receive a cash prize, a featured product or products, or other prize that is related to health, wellness or weight loss. Optimally, prize product(s) would be provided by an advertiser on the show, thereby giving promotion to the advertiser and their product(s).

As a result of modern technology, there is the possibility of a participant(s) attempting to gain an advantage by using a device such as a computer or smartphone while shopping to determine which product(s) would be best to select. This could especially be a problem if one of the metrics that would determine the winner of a prize is which participant selects the healthiest alternative product(s) to what is considered an un-healthy choice. The easiest solution to this problem would be to not disclose what the show is about to participants prior to the participants agreeing to be on the show. There is also the ability for the host(s) and/or the technical coordinator (or even a dedicated observer) to accompany the participant(s) and the host(s) while shopping, or being present in the participant's dwelling when going through the products the participant(s) previously purchased, and ensuring the participant(s) do not use any type of device with internet accessibility. In another scenario, the participant(s) are not told what the show is about and are secretly observed when the participant(s) are shopping. The host(s), who is located remotely and out of sight of the participant(s) will comment on and judge the participant(s) selections. The host(s) will also offer suggestions for alternative product(s) based on what is observed. At the end of the show, after multiple participant(s) have been shopping while being observed by the host(s), the participant(s) who made the best (i.e., healthiest) choices would be deemed the winner and could win a prize. An additional means of ensuring the participants are not attempting to gain an advantage by going to a store and pre-selecting the healthiest products (even though these products are not what the participant(s) would normally choose) would be to have a group of participants sequested at a location. The participants could be monitored and if desired, the participants could not use their computers, smartphone or other device to access the internet. To add to the show's theme, the sequester location could be stocked with food and other products, some of the food items and other products would be healthy while others would be un-healthy. The host(s) or panel could then comment on, judge and offer suggestions based on what the participants choose to eat and use. The shows production item could stock the sequester location, or the participants, along with the host(s) could go shopping to purchase products for the locations.

While the invention has been described in detail and pictorially shown in the accompanying drawings it is not to be limited to such details, since many changes and modifications may be made to the invention without departing from the spirit and the scope thereof. Hence, it is described to cover any and all modifications and forms which may come within the language and scope of the claims.

1. A method for conducting a health, wellness and weight loss reality show, comprising:
selecting, by at least one participant products for purchase from a collaborating store,
accompanying, by at least one host, the at least one participant while the participant selects the products in the collaborating store,
first receiving and transmitting, by at least one participant transceiver, first audio and visual communications to a production facility,
second receiving and transmitting, by at least one host transceiver,
second audio and visual communication to a production facility,
processing with computer software the first and second audio and visual communications from the at least one participant and at least one host for broadcast on a viewing medium, and
commenting on and judging, by the at least one host, the selections of the participant from a health, wellness and weight loss potential and perspective.

2. The method for conducting a health, wellness and weight loss reality show in claim 1, wherein the store comprises a market.

3. The method for conducting a health, wellness and weight loss reality show in claim 1, wherein the market comprises a chain supermarket.

4. The method for conducting a health, wellness and weight loss reality show in claim 1, wherein the store comprises a big box retailer.

5. The method for conducting a health, wellness and weight loss reality show in claim 1, wherein the store comprises a drug store.

6. The method for conducting a health, wellness and weight loss reality show in claim 1, wherein the selecting products are selected from the group consisting of fresh food and vegetables, canned goods, frozen food items, dairy items, beverages, and personal health and hygienic products.

7. The method for conducting a health, wellness and weight loss reality show in claim 1, further comprising granting, by the collaborating store to the at least one participant and as least one host, permission to transmit and record the participant in-store product selections and the host's commenting and judging the product selections.

8. The method for conducting a health, wellness and weight loss reality show in claim 1, wherein the transceiver comprises a video camera and audio recording device.

9. The method for conducting a health, wellness and weight loss reality show in claim 1, further comprising broadcasting the at least one participant and at least one host interactions to a live audience.

10. The method for conducting a health, wellness and weight loss reality show in claim 1, further comprising manipulating with the computer software, the video and audio for optimum viewing quality; and
editing with the computer software, the broadcast within an alotted time period.

11. The method for conducting a health, wellness and weight loss reality show in claim 1, wherein the viewing medium is selected from the group consisting of a television show, a television series, an internet show, a podcast and a recorded digital medium that is played back on a dedicated playing device.

12. A method for conducting a health, wellness and weight loss reality show comprising:
purchasing, by at least one participant, products from a store,
commenting on and judging, by at least one host, the purchased products,
transceiving, by at least one transceiver, audio and visual communication with a production facility, and
processing, by a computer operating software, the communications from the at least one participant and the at least one host for broadcast on a viewing medium, with the host commenting on and judging the purchased products from a health, wellness and weight loss potential and perspective.

13. The method for conducting a health, wellness and weight loss reality show in claim 12, wherein the host commenting on and judging the purchased products occurs substantially immediately after the at least one participant has left a location where the products were purchased.

14. The method for conducting a health, wellness and weight loss reality show in claim 12, further comprising commenting or judging, by the host, purchased products that are already within the dwelling of the at least one participant.

15. The method for conducting a health, wellness and weight loss reality show in claim 12, further comprising selecting the at least one participant from the group consisting of an individual, a couple, a family and a combination of related or non-related people.

16. The method for conducting a health, wellness and weight loss reality show in claim 12, further comprising selecting the products from the group consisting of fresh food and vegetables, canned goods, frozen food items, dairy items, beverages, and personal health and hygienic products.

17. The method for conducting a health, wellness and weight loss reality show in claim 12, wherein the transceiver comprises a video camera and audio recording device.

18. The method for conducting a health, wellness and weight loss reality show in claim 12, further comprising broadcasting the at least one participant and at least one host interactions to a live audience.

19. The method for conducting a health, wellness and weight loss reality show in claim 12, wherein the software that processes transceived communication is configured to manipulate the video and audio for optimum viewing quality and to edit the communications for broadcast within an alotted time period.

20. The method for conducting a health, wellness and weight loss reality show in claim 12, wherein the viewing medium is selected from the group consisting of a television show, a television series, and internet show, a podcast and a recorded digital medium that is played back on a dedicated playing device.
